SEQUENCE LISTING (2 sequences)

SEQ ID NO: 1 - protein, 120 aa, Artificial Sequence
  Synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVS

SEQ ID NO: 2 - protein, 109 aa, Artificial Sequence
  Synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRT

(19) United States
(12) Patent Application Publication
(10) Pub. No.: US 2006/0147450 A1
(43) Pub. Date: Jul. 6, 2006

(54) Methods for treating cardiac arrhythmia and preventing death due to cardiac arrhythmia using ngf antagonists

(76) Inventor: David L Shelton, Oakland, CA (US)

Correspondence Address: MORRISON & FOERSTER LLP, 755 PAGE MILL RD, PALO ALTO, CA, 94304-1018, US

(21) Appl. No.: 10/529,835

(22) PCT Filed: Oct. 6, 2003
(86) PCT No.: PCT/US03/31631

§ 371 Date: Oct. 13, 2005

Related U.S. Application Data
(60) Provisional application No. 60/415,989, filed on Oct. 4, 2002.

(51) Int. Cl. A61K 39/395

(52) U.S. Cl. 424/145.1

(57) ABSTRACT

This invention relates to the field of cardiac disease. More specifically, the invention relates to methods using an NGF antagonist for treating and preventing cardiac arrhythmia and methods of preventing death due to cardiac arrhythmia.

### CROSS-REFERENCE TO RELATED APPLICATIONS

This application claims the priority benefit of the provisional patent application U.S. Ser. No. 60/415,989, filed Oct. 4, 2002, which is incorporated herein by reference in its entirety.

### STATEMENT REGARDING FEDERALLY SPONSORED RESEARCH OR DEVELOPMENT

Not applicable.

### FIELD OF THE INVENTION

This invention relates to the field of cardiac disease. More specifically, the invention relates to methods for treating and preventing cardiac arrhythmia and methods of preventing death due to cardiac arrhythmia.

### BACKGROUND OF THE INVENTION

Sudden cardiac death (SCD) is the most common lethal manifestation of heart diseases, and accounts for >64% of all cardiac disease deaths. Zheng et al., Circulation 104:2158-2163 (2001); Priori et al., European Heart J. 22:1374-1450 (2001). SCD is the result of unresuscitated cardiac arrest, which may accompany almost all known heart diseases, such as myocardial infarction, ischemic heart disease, coronary artery disease, mitral insufficiency, and hypertrophic cardiomyopathy. While the underlying mechanism of SCD is not well understood, in most cases, the direct cause of the SCD is ventricular fibrillation (“VF”), which may be triggered by ventricular tacchycardia (“VT”).

VT and VF are different types of cardiac arrhythmias. VT is abnormally fast ventricular heart rhythm which is, by itself, typically not fatal. VF is a chaotic ventricular heart rhythm which produces little or no net blood flow from the heart, such that there is little or no net blood flow to the brain and other vital organs. VF, if not terminated, results in death. In 75% of SCD cases, the victim has a previous myocardial infarction (“MI”), i.e., the patient had a previous heart attack caused by blockage of a portion of the coronary artery which supplies blood to the heart muscle. As a result of the blockage, a portion of the heart muscle does not receive blood and therefore becomes scarred and diseased. Numerous VT episodes can occur subsequent to the occurrence of MI, and these VT episodes may eventually lead or transition to VF resulting in SCD of the patient.

Hearts are innervated by both sympathetic and parasympathetic nerves. The sympathetic and parasympathetic nerves work together to assure that the four chambers of the heart (the atria and ventricles) contract at a rate appropriate to physiological conditions. Cardiac sympathetic innervation comes from postganglionic fibers that arise from both cervical and thoracic sympathetic ganglia. The sympathetic innervation to the ventricles arises from the superficial and deep cardiac plexuses via the right and left coronary plexuses. The sympathetic nerves are distributed to the myocardium in superficial layers, and penetrate the myocardium along with the coronary arteries. The parasympathetic preganglionic fibers make synaptic connections with ganglion cells in the cardiac plexus or within intracardiac terminal ganglia.

Substantial animal and clinical data implicate the sympathetic nervous system in arrhythmogenesis and SCD. Podrid et al., Circulation 82 (suppl 1):I-130 thru I-113 (1990); Vanoli & Schwartz, Basic Res. Cardiol. 85:305-321 (1990). For example, introduction of agents capable of counteracting the consequences of sympathetic stimulation, such as calcium channel blockers and beta-blockers, reduces the occurrence of ventricular tachycardia and ventricular fibrillation. See, e.g., Priori et al., Am. Heart J. 116:37 (1988). Furthermore, sympathetic scintigraphy demonstrated both denervation and reinnervation by sympathetic nerves after MI. See, e.g., Stanton et al., J. Am. Coll. Cardiol. 14:1519-1526 (1989); Minardo et al., Circulation 78:1008-1019 (1988). However, the functional significance of the sympathetic innervation or denervation and the underlying cause of arrhythmogenesis and SCD are poorly understood.

Nerve growth factor (NGF) was the first neurotrophin to be identified, and its role in the development and survival of both peripheral and central neurons has been well characterized. NGF has been shown to be a critical survival and maintenance factor in the development of peripheral sympathetic and embryonic sensory neurons and of basal forebrain cholinergic neurons. Smeyne et al., Nature 368:246-249 (1994); Crowley et al., Cell 76:1001-1011 (1994). NGF upregulates expression of neuropeptides in sensory neurons (Lindsay and Harmer, Nature 337:362-364 (1989)) and its activity is mediated through two different membrane-bound receptors, the TrkA receptor and the p75 common neurotrophin receptor (sometimes termed “high affinity” and low affinity” NGF receptors, respectively). Chao et al., Science 232-518-521 (1986). For review on NGF, see Huang et al., Annu. Rev. Neurosci. 24:677-736 (2001); Bibel et al., Genes Dev. 14:2919-37 (2000). The crystal structure of NGF and NGF in complex with the trkA receptor has been determined. See Nature 254:411 (1991); Nature 401:184-88 (1996).

Whether NGF plays a causative or a protective role with respect to cardiac arrhythmia is subject to debate. NGF level is elevated in the myocardium of an individual predisposed to cardiac arrhythmia. See, e.g., Hiltunen, et al., J. Pathol. 194(2): 274-53 (2001); Gu et al., Circulation 90(Suppl I):I-249 (1994). But the exact role of NGF in arrhythmogenesis remains poorly understood. In one study, Cao et al. introduced exogenous NGF to the left sympathetic stellate ganglion in dogs following an MI and an AV block, creating an animal model with high incidence of spontaneous VT, VF, and SCD. Cao et al., Circulation Research 86:816-821 (2000). Cao et al. found that NGF infusion augmented sympathetic nerve sprouting, and proposed the so-called “nerve sprouting hypothesis,” i.e., that MI results in nerve and tissue injury, followed by sympathetic nerve sprouting and regional (heterogeneous) myocardial hyperinnervation. They hypothesized that the electrical coupling between the augmented sympathetic nerve sprouting with the electrically remodeled myocardium results in VT, VF, and SCD. Cao et al., Circulation Research 86:816-821 (2000); Chen et al., Cardiovascular Research 50:409-416 (2001); PCT WO 01/62334. On the other hand, some researchers believe that decreased sympathetic innervation or activity may facilitate malignant cardiac arrhythmogenesis, and that NGF plays a protective role against sympathetic denervation or dysfunction. Schmid, et al., Diabetes 48:603-608 (1999); Abe et al., Circulation 95(1): 213-220 (1997). For example, studies done by Abe et al. showed that NGF exogenously infused over a short period of time, as well as endogenously released NGF, protect against acute post-ischemic neural dysfunction of sympathetic cardiac innervation. Abe et al. speculated that NGF altered the sympathetic reactivity indirectly, possibly by increasing the number of functional sodium channels through cAMP-dependent protein kinase. Abe et al., Circulation 95(1):213-220 (1997).

Until now, the primary therapy for potentially fatal cardiac arrhythmia is to implant pacemakers or cardioverter-defibrillators (ICD) in patients at high risk of developing such arrhythmias. The implanted devices are expensive, require frequent replacement and may be extremely painful to the patient. Furthermore, most patients have not experienced a preceding symptomatic arrhythmic event, and therefore such expensive and invasive therapy is not generally justified. Beta-blocking agents have also been used to treat patients with arrhythmia in which the sympathetic nerve system is involved. However, these agents have side effects caused by beta-blocking actions, such as depression of cardiac function, induction of bronchial asthmatic attack, and hypoglycemic seizures. No current therapy acts to prevent or reverse some or all of the underlying pathology that gives rise to a cardiac arrhythmia.

Accordingly, there is a great need for new therapeutic drugs for the treatment and prevention of cardiac arrhythmia.

All references cited herein, including patent applications and publications, are incorporated by reference in their entirety.

### BRIEF SUMMARY OF THE INVENTION

The present invention provides methods for treating NGF-associated cardiac arrhythmia comprising administering an NGF antagonist that blocks, suppresses and/or reduces (including significantly) NGF activity. We believe NGF-stimulated inappropriate sympathetic nerve function and/or nerve fiber growth occurs following MI or other triggering events.

Accordingly, the present invention provides methods of treating, preventing, and/or reducing incidence of NGF-associated cardiac arrhythmia in an individual comprising administering an effective amount of an NGF antagonist. The invention also provides methods of treating, preventing, and/or reducing incidence of a cardiac arrhythmia in an individual at risk of developing the cardiac arrhythmia comprising administering an effective amount of an NGF antagonist. In another aspect, the invention provides methods of preventing or reducing risk of death due to cardiac arrhythmia comprising administering an effective amount of an NGF antagonist to an individual at risk of developing the cardiac arrhythmia. The invention also provides methods of enhancing cardiac function in individuals in need thereof (such as an individual with MI) comprising administering an effective amount of an NGF antagonist.

In some embodiments, the cardiac arrhythmia comprises one or more of sustained VT, non-sustained VT, VF, ventricular premature beats, ventricular flutter, and atrial tachyarrhythmia (such as atrial fibrillation and atrial flutter). In some embodiments, the cardiac arrhythmia is associated with SCD. Exemplary cardiac arrhythmias associated with SCD include, but are not limited to, VT and VF. In other embodiments, the cardiac arrhythmia is associated with sympathetic hyperinnervation (increased sympathetic nerve fiber growth) or increased sympathetic nerve function. In some embodiments, the individual having or at risk for a cardiac arrhythmia is: an individual who has survived an episode of cardiac arrest, an individual who has been resuscitated from near-fatal ventricular fibrillation or ventricular tacchycardia, an individual who has suffered at least one episode of myocardial infarction, and/or an individual who has suffered acute or chronic ischemic heart disease.

An NGF antagonist suitable for use in the methods of the invention is any agent that can directly or indirectly result in decreased NGF biological activity. In some embodiments, an NGF antagonist (e.g., an antibody) binds (physically interacts with) NGF, binds to an NGF receptor (such as trkA receptor or p75), and/or reduces (impedes and/or blocks) downstream NGF receptor signaling (e.g., inhibitors of kinase signaling). Accordingly, in some embodiments, an NGF antagonist binds (physically interacts with) NGF. In other embodiment, an NGF antagonist binds to an NGF receptor (such as trkA receptor or p75). In other embodiments, an NGF antagonist reduces (impedes and/or blocks) downstream NGF receptor signaling (e.g., inhibitors of kinase signaling). In other embodiments, an NGF antagonist inhibits (reduces) NGF synthesis and/or production (release). In some embodiments, the NGF antagonist is selected from the following: an anti-NGF antibody, an anti-sense molecule directed to an NGF (including an anti-sense molecule directed to a nucleic acid encoding NGF), an anti-sense molecule directed toward an NGF receptor (such as trkA and/or p75), an NGF inhibitory compound, an NGF structural analog, a dominant-negative mutation of a TrkA receptor that binds an NGF, a TrkA immunoadhesin, an anti-TrkA antibody, an anti-p75 antibody and a kinase inhibitor. In other embodiments, the NGF antagonist is an anti-NGF antibody. In other embodiments, the anti-NGF antibody (interchangeably termed “NGF antibody”) recognizes (binds) human NGF. In other embodiments, the anti-NGF antibody specifically binds human NGF. In still other embodiments, the anti-NGF antibody is humanized (such as antibody E3 described herein). In some embodiments, the anti-NGF antibody is antibody E3 (as described herein). In other embodiments, the anti-NGF antibody comprises one or more CDR(s) of antibody E3 (such as one, two, three, four, five, or, in some embodiments, all six CDRs from E3). In other embodiments, the antibody is human. In other embodiments, the antibody comprises a modified constant region, such as a constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT/GB99/01441; and/or UK patent application No. 9809951.8.

The binding affinity of an anti-NGF antibody to NGF (such as hNGF) can be about 0.10 to about 0.80 nM, about 0.15 to about 0.75 nM and about 0.18 to about 0.72 nM. In one embodiment, the binding affinity is between about 2 pM and 22 pM. In some embodiment, the binding affinity is about 10 nM. In other embodiments, the binding affinity is less than about 10 nM. In other embodiments, the binding affinity is about 0.1 nM. In other embodiments, the binding affinity is less than about 0.1 nM. In other embodiments, the binding affinity is any of about 100 nM, about 50 nM, about 10 nM, about 1 nM, about 500 pM, about 100 pM, or about 50 pM to any of about 2 pM, about 5 pM, about 10 pM, about 15 pM, about 20 pM, or about 40 pM. In some embodiments, the binding affinity is any of about 100 nM, about 50 nM, about 10 nM, about 1 nM, about 500 pM, about 100 pM, or about 50 pM, or less than about 50 pM. In some embodiments, the binding affinity is less than any of about 100 nM, about 50 nM, about 10 nM, about 1 nM, about 500 pM, about 100 pM, or about 50 pM. In still other embodiments, the binding affinity is about 2 pM, about 5 pM, about 10 pM, about 15 pM, about 20 pM, about 40 pM, or greater than about 40 pM. As is well known in the art, binding affinity can be expressed as K_{D}, or dissociation constant, and an increased binding affinity corresponds to a decreased K_{D}. The binding affinity of anti-NGF mouse monoclonal antibody 911 (Hongo et al., Hybridoma 19:215-227 (2000) to human NGF is about 10 nM, and the binding affinity of humanized anti-NGF antibody E3 (described herein) to human NGF is about 0.1 nM.

Administration can be by any means known in the art, including, for example, orally, intravenously, subcutaneously, intraarterially (such as via a coronary artery), intramuscularly, intracardially, intraspinally, intrathoracicly, intraperitoneally, intraventricularly, sublingually, via inhalation, injection into a sympathetic ganglion and/or trunk, and transdermally. In some embodiments, the NGF antagonist is an anti-NGF antibody, and administration is by one or more of the following means: intravenously, subcutaneously, via inhalation, intrarterially, intramuscularly, intracardially, intraventricularly, and intraperitoneally. Administration may be systemic (e.g. intravenously), or localized. Administration may be acute or chronic.

In another aspect, the invention provides compositions and kits comprising an NGF antagonist for use in any of the methods of the invention.

### DETAILED DESCRIPTION OF THE INVENTION

The present invention provides methods for treating NGF-associated cardiac arrhythmia comprising administering NGF antagonist that blocks, suppresses and/or reduces (including significantly) NGF activity. We believe NGF-stimulated inappropriate sympathetic nerve function and/or nerve fiber growth occurs following MI or other triggering events.

Accordingly, the present invention provides methods of treating, preventing, and/or reducing incidence of a NGF-associated cardiac arrhythmia in an individual comprising administering an effective amount of an NGF antagonist. The invention also provides methods of treating, preventing, and/or reducing incidence of a cardiac arrhythmia in an individual at risk of developing the cardiac arrhythmia comprising administering an effective amount of an NGF antagonist. In another aspect, the invention provides methods of preventing or reducing risk of death due to cardiac arrhythmia comprising administering an effective amount of an NGF antagonist. The invention also provides methods of enhancing cardiac function in individuals in need thereof (such as an individual with MI or history of MI) comprising administering an effective amount of an NGF antagonist.

Cardiac arrhythmias are well known in the art. Exemplary arrhythmias include one or more of the following: sustained VT, non-sustained VT, VF, ventricular premature beats, ventricular flutter, and atrial tachyarrhythmia (including atrial fibrillation and atrial flutter). In some embodiments, the cardiac arrhythmia is associated with increased incidence of SCD. Exemplary cardiac arrhythmias associated with SCD include, but are not limited to, VT and VF. In other embodiments, the cardiac arrhythmia is associated with sympathetic hyperinnervation (such as increased sympathetic nerve fiber growth) and/or sympathetic hyperactivity In some embodiments, the individual having or at risk for a cardiac arrhythmia is: an individual who has survived an episode of cardiac arrest, an individual who has been resuscitated from near-fatal ventricular fibrillation or ventricular tachycardia, an individual who has suffered at least one episode of myocardial infarction, and/or an individual who has suffered acute or chronic ischemic heart disease.

An NGF antagonist suitable for use in the methods of the invention is any agent that can directly or indirectly result in decreased NGF biological activity. In some embodiments, an NGF antagonist (e.g., an antibody) binds (physically interacts with) NGF, binds to an NGF receptor (such as trkA receptor or p75), and/or reduces (impedes and/or blocks) downstream NGF receptor signaling (e.g., inhibitors of kinase signaling). Accordingly, in some embodiments, an NGF antagonist binds (physically interacts with) NGF. In other embodiment, an NGF antagonist binds to an NGF receptor (such as trkA receptor or p75). In other embodiments, an NGF antagonist reduces (impedes and/or blocks) downstream NGF receptor signaling (e.g., inhibitors of kinase signaling). In other embodiments, an NGF antagonist inhibits (reduces) NGF synthesis and/or production (release). In some embodiments, the NGF antagonist is selected from the following: an anti-NGF antibody, an anti-sense molecule directed to an NGF (including an anti-sense molecule directed to a nucleic acid encoding NGF), an anti-sense molecule directed toward an NGF receptor (such as trkA and/or p75), an NGF inhibitory compound, an NGF structural analog, a dominant-negative mutation of a TrkA receptor that binds an NGF, a TrkA immunoadhesin, an anti-TrkA antibody, an anti-p75 antibody and a kinase inhibitor. In other embodiments, the NGF antagonist is an anti-NGF antibody. In other embodiments, the NGF antibody recognizes human NGF. In still other embodiments, the anti-NGF antibody is a humanized antibody (such as antibody E3 described herein). In some embodiments, the anti-NGF antibody is antibody E3 (as described herein). In other embodiments, the anti-NGF antibody comprises one or more CDR(s) of antibody E3 (such as one, two, three, four, five, or, in some embodiments, all six CDRs from E3). In other embodiments, the antibody is human. In other embodiments, the antibody comprises a modified constant region, such as a constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT/GB99/01441; and/or UK patent application No. 9809951.8.

The binding affinity of an anti-NGF antibody to NGF (such as hNGF) can be about 0.10 to about 0.80 nM, about 0.15 to about 0.75 nM and about 0.18 to about 0.72 nM. In one embodiment, the binding affinity is between about 2 pM and 22 pM. In some embodiment, the binding affinity is about 10 nM. In other embodiments, the binding affinity is less than about 10 nM. In other embodiments, the binding affinity is about 0.1 nM. In other embodiments, the binding affinity is less than about 0.1 nM. In other embodiments, the binding affinity is any of about 100 nM, about 50 nM, about 10 nM, about 1 nM, about 500 pM, about 100 pM, or about 50 pM to any of about 2 pM, about 5 pM, about 10 pM, about 15 pM, about 20 pM, or about 40 pM. In some embodiments, the binding affinity is any of about 100 nM, about 50 nM, about 10 nM, about 1 nM, about 500 pM, about 100 pM, or about 50 pM, or less than about 50 pM. In some embodiments, the binding affinity is less than any of about 100 nM, about 50 nM, about 10 nM, about 1 nM, about 500 pM, about 100 pM, or about 50 pM. In still other embodiments, the binding affinity is about 2 pM, about 5 pM, about 10 pM, about 15 pM, about 20 pM, about 40 pM, or greater than about 40 pM. As is well known in the art, binding affinity can be expressed as K_{D}, or dissociation constant, and an increased binding affinity corresponds to a decreased K_{D}. The binding affinity of the anti-NGF mouse monoclonal antibody 911 (also called “Mab 911”) to human NGF is about 10 nM, and the binding affinity of the humanized anti-NGF antibody E3 (described herein) to human NGF is about 0.1 nM.

Administration can be, for example, orally, intravenously, subcutaneously, intraarterially, intramuscularly, intracardially, intraspinally, intrathoracicly, intraperitoneally, intraventricularly, sublingually, or transdermally. In some embodiments, the NGF antagonist is an anti-NGF antibody, and administration is by one or more of the following means: intravenously, subcutaneously, via inhalation, intrarterially, intramuscularly, intracardially, intraventricularly, and intraperitoneally. Administration may be systemic, e.g. intravenously or localized.

In another aspect, the invention provides compositions and kits comprising an NGF antagonist for use in any of the methods of the invention.

I. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

II. Definitions

An “antibody” (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (ScFv), mutants thereof, fusion proteins comprising an antibody portion, humanized antibodies, chimeric antibodies, diabodies linear antibodies, single chain antibodies, multispecific antibodies (e.g., bispecific antibodies) and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′)2, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody. In some instances, framework region (FR) residues or other residues of the human immunoglobulin replaced by corresponding non-human residues. Furthermore, humanized antibodies may comprise residues which are not found in the recipient antibody or in the donor antibody.

As used herein, the term “nerve growth factor” and “NGF” refers to nerve growth factor and variants thereof that retain at least part of the activity of NGF. As used herein, NGF includes all mammalian species of native sequence NGF, including human, canine, feline, equine, or bovine.

“NGF receptor” refers to a polypeptide that is bound by or activated by NGF. NGF receptors include the TrkA receptor and the p75 receptor of any mammalian species, including, but are not limited to, human, canine, feline, equine, primate, or bovine.

An “NGF antagonist” refers to any molecule that blocks, suppresses or reduces (including significantly) NGF biological activity, including downstream pathways mediated by NGF signaling, such as receptor binding and/or elicitation of a cellular response to NGF. The term “antagonist” implies no specific mechanism of biological action whatsoever, and is deemed to expressly include and encompass all possible pharmacological, physiological, and biochemical interactions with NGF whether direct or indirect, or whether interacting with NGF, its receptor, or through another mechanism, and its consequences which can be achieved by a variety of different, and chemically divergent, compositions. Exemplary NGF antagonists include, but are not limited to, an anti-NGF antibody, an anti-sense molecule directed to an NGF (including an anti-sense molecule directed to a nucleic acid encoding NGF), an NGF inhibitory compound, an NGF structural analog, a dominant-negative mutation of a TrkA receptor that binds an NGF, a TrkA immunoadhesin, an anti-TrkA antibody, an anti-p75 antibody, and a kinase inhibitor. For purpose of the present invention, it will be explicitly understood that the term “antagonist” encompass all the previously identified terms, titles, and functional states and characteristics whereby the NGF itself, an NGF biological activity (including but not limited to its ability to ability to stimulate sympathetic hyperinnervation), or the consequences of the biological activity, are substantially nullified, decreased, or neutralized in any meaningful degree. In some embodiments, an NGF antagonist (e.g., an antibody) binds (physically interacts with) NGF, binds to an NGF receptor (such as trkA receptor or p75 receptor), reduces (impedes and/or blocks) downstream NGF receptor signaling, and/or inhibits (reduces) NGF synthesis, production or release. Examples of types of NGF antagonists are provided herein.

As used herein, an “anti-NGF antibody” refers to an antibody which is able to bind to NGF and inhibit NGF biological activity and/or downstream pathway(s) mediated by NGF signaling.

A “TrkA immunoadhesin” refers to a soluble chimeric molecule comprising a fragment of a TrkA receptor, for example, the extracellular domain of a TrkA receptor and an immunoglobulin sequence, which retains the binding specificity of the TrkA receptor and is capable of blocking the biological activity of NGF.

“Biological activity” of NGF generally refers to the ability to bind NGF receptors and/or activate NGF receptor signaling pathways. Without limitation, a biological activity includes any one or more of the following: the ability to bind an NGF receptor (such as p75 and/or trkA); the ability to promote trkA receptor dimerization and/or autophosphorylation; the ability to activate an NGF receptor signaling pathway; the ability to promote cell differentiation, proliferation, survival, growth and other changes in cell physiology, including (in the case of neurons, including peripheral and central neuron) change in neuronal morphology, neuronal activity, synaptogenesis, synaptic function, neurotransmitter and/or neuropeptide release, and regeneration following damage; the ability to promote cardiac sympathetic hyperinnervation; and the ability to generate, cause, trigger and/or aggravate one or more symptoms of cardiac arrhythmia

The term “epitope” is used to refer to binding sites for (monoclonal or polyclonal) antibodies on protein antigens.

An “NGF-associated cardiac arrhythmia” (interchangeably termed “cardiac arrhythmia”) refers to a disturbance of the electrical activity of the heart, in which an abnormality in heart rate or heart rhythm is manifested. As used herein, NGF is associated with these cardiac arrhythmias, meaning that one or more cardiac arrhythmia results, either directly or indirectly, from NGF activity or hyperactivity, or NGF plays a role, at least, as a causative and/or contributory factor, in these arrhythmia(s). This is indicated by using an NGF antagonist (such as an anti-NGF antibody) to treat the arrhythmia, or an arrhythmia for which administration of an NGF antagonist is desirable. As is evident to one of ordinary skill in the art, administering (or the desirability of administering) an NGF antagonist for effectiveness in the context of cardiac arrhythmia (including risk of cardiac arrhythmia) indicates the arrhythmia is NGF associated. In other embodiments, the arrhythmia involves the activity of the sympathetic nervous system. In some embodiments, the arrhythmia results from sympathetic hyperinnervation (increased sympathetic nerve fiber growth). Examples of cardiac arrhythmia include, but are not limited to, sustained ventricular tachycardia, non-sustained ventricular tachycardia, ventricular fibrillation, ventricular premature beats, ventricular flutter, and atrial tachyarrhythmia (such as atrial fibrillation and atrial flutter). The cardiac arrhythmia can be accompanied by and/or precede sudden cardiac death. It is understood that an individual can possess one or more types of arrhythmia

As used herein, “sudden cardiac death” or “SCD” refers to the sudden, abrupt loss of heart function in a person who may or may not have diagnosed heart disease, but in whom the time and mode of death occur unexpectedly. SCD generally occurs within one hour of the onset of acute disease or symptoms, but it is understood that the underlying diseases/symptoms and/or cardiac dysfunction may have been present for longer than one hour.

As used herein, “ventricular tachycardia” (“VT”) refers to abnormal accelerated ventricular rhythm. Ventricular tachycardia may result in fainting, low blood pressure, shock, or sudden cardiac death, and has the potential of degrading to a more serious ventricular fibrillation.

As used herein, “ventricular fibrillation” (“VF”) refers a disorganized chaotic contraction of the ventricles that significantly decreases blood ejection from the ventricles. Generally, during ventricular fibrillation, the patient is or becomes unconscious and can die if no immediate treatment is undertaken.

As used herein, “myocardial infarction” (interchangeably termed “heart attack”), refers to damage that occurs to the heart when one of the coronary arteries or its branches becomes occluded.

As used therein, “sympathetic hyperinnervation” refers to undesired sympathetic nerve sprouting or nerve fiber growth in the myocardium.

As used herein, treatment” is an approach for obtaining beneficial or desired clinical results. For purposes of this invention, beneficial or desired results, including clinical results include, but are not limited to, one or more of the following: alleviation or elimination of one or more symptoms associated with a cardiac arrhythmia, amelioration of a cardiac arrhythmia, palliation of a cardiac arrhythmia, stabilized (i.e., not worsening) state of a cardiac arrhythmia, delaying, suppressing or preventing the occurrence/recurrence of cardiac arrhythmia, delaying, suppressing, or preventing the development of cardiac arrhythmia, remission (whether partial or total) or reduction of incidence of cardiac arrhythmia and/or symptoms associated with cardiac arrhythmia and/or sudden cardiac death. In some embodiments, the cardiac arrhythmia is an arrhythmia associated with increased incidence of sudden cardiac death.

“Reducing incidence” of arrhythmia means any of reducing severity (which can include reducing need for and/or amount of (e.g., exposure to) other drugs and/or therapies generally used for this conditions, including, for example, beta blockers and ICD), duration, and/or frequency (including, for example, delaying or increasing time to arrhythmia in an individual). As is understood by those skilled in the art, individuals may vary in terms of their response to treatment, and, as such, for example, a “method of reducing incidence of arrhythmia in an individual” reflects administering the NGF antagonist described herein based on a reasonable expectation that such administration may likely cause such a reduction in incidence in that particular individual.

“Ameliorating” a cardiac arrhythmia or one or more symptoms of cardiac arrhythmia means a lessening or improvement of one or more symptoms of a cardiac arrhythmia as compared to not administering an NGF antagonist. “Ameliorating” also includes shortening or reduction in duration of a symptom.

“Palliating” a cardiac arrhythmia or one or more symptoms of a cardiac arrhythmia means lessening the extent of undesirable clinical manifestations of a cardiac arrhythmia in an individual or population of individuals treated with an NGF antagonist in accordance with the invention.

As used therein, “delaying” the development of cardiac arrhythmia means to defer, hinder, slow, retard, stabilize, and/or postpone progression of a cardiac arrhythmia. This delay can be of varying lengths of time, depending on the history of the disease and/or individuals being treated. As is evident to one skilled in the art, a sufficient or significant delay can, in effect, encompass prevention, in that the individual does not develop arrhythmia (or die due to SCD). A method that “delays” development of the symptom is a method that reduces probability of developing the symptom in a given time frame and/or reduces extent of the disease in a given time frame, when compared to not using the method. Such comparisons are typically based on clinical studies, using a statistically significant number of subjects.

“Development” or “progression” of a cardiac arrhythmia means initial manifestations and/or ensuing progression of the disorder. Development of cardiac arrhythmia can be detectable and assessed using standard clinical techniques as described herein. However, development also refers to disease progression that may be undetectable. For purpose of this invention, development or progression refers to the biological course of the disease state. Progression includes occurrence of SCD. “Development” includes occurrence, recurrence, and onset. As used herein “onset” or “occurrence” of cardiac arrhythmia includes initial onset and/or recurrence.

As used herein, an individual “at risk of developing a cardiac arrhythmia” is an individual who is considered more likely to develop cardiac arrhythmia than an individual in the general population. An individual “at risk” may or may not have detectable disease, and may or may not have displayed detectable disease prior to the treatment methods described therein. “At risk” denotes that an individual who is determined to be more likely to develop a symptom based on conventional risk assessment methods or has one or more risk factors that correlate with development of cardiac arrhythmia described therein. An individual having one or more of these risk factors has a higher probability of developing cardiac arrhythmia than an individual without these risk factors. Examples (i.e., categories) of risk groups are discussed below.

Depending on the basis and context of assessment of risk, the time frame within which probability of cardiac arrhythmia development, progression, and/or onset would more likely than not occur would vary. For instance, for individuals with previous myocardial infarction, the risk of occurrence is typically measured within a year. For an individual who is considered at risk due to, for example, genetic or hereditary considerations, the risk of occurrence can be measured in a longer time frame, including the expected lifetime of the individual.

An individual with “low risk” is one who is not considered “at risk”.

An “effective amount” is an amount sufficient to effect beneficial or desired clinical results including clinical results or delaying the onset of the disease. An effective amount can be administered in one or more administrations. For purposes of this invention, an effective amount of an NGF antagonist described herein is an amount sufficient to treat, ameliorate, stabilize, reverse, slow or delay progression of or prevent a cardiac arrhythmia or SCD.

An “individual” is a vertebrate, preferably a mammal, more preferably a human. Mammals include, but are not limited to, farm animals, sport animals, pets, primates, horses, cats, dogs, mice and rats.

“Comprising” means including, in accordance with well established principles of patent law.

As used herein, the singular form “a”, “an”, and “the” includes plural references unless indicated otherwise. For example, “an” antibody includes one or more antibodies and “a symptom” means one or more symptoms.

III. Methods of the Invention

With respect to all methods described herein, reference to NGF antagonists also includes compositions comprising one or more of these agents. These compositions may further comprise suitable excipients, such as pharmaceutically acceptable excipients including buffers, which are well known in the art. The present invention can be used alone or in combination with other conventional methods of treatment, such as a treatment using a cardioverter-defibrillator.

Methods for Treating NGF-Associated Cardiac Arrhythmia

The present invention encompasses methods of treating a NGF-associated cardiac arrhythmia in an individual comprising administering an effective amount of an NGF antagonist. In some embodiments, the cardiac arrhythmia comprises one or more of: sustained VT, non-sustained VT, Phase I VT, Phase II VT, VF, ventricular premature beats, ventricular flutter, and/or atrial tachyarrhythmia (including atrial fibrillation and atrial flutter). In other embodiments, the cardiac arrhythmia is associated with SCD. Exemplary cardiac arrhythmias associated with SCD include, but are not limited to, VT and VF. In other embodiments, the cardiac arrhythmia is associated with sympathetic hyperinnervation, and/or increased sympathetic nerve function (hyperactivity).

As will be understood by those skilled in the art, a cardiac arrhythmia can be an isolated event, or more than one arrhythmia can occur in one individual simultaneously. One or more kind of arrhythmia can also occur sequentially. The arrhythmia may be intermittent. Different kinds of cardiac arrhythmias can be causally inter-related. For example, a VT can trigger the occurrence of a VF. It should be generally understood that the present invention encompasses methods of treatment of cardiac arrhythmias in any of those situations.

Cardiac arrhythmia can be associated with the occurrence of another cardiac disorder or “precursor disease”, such as MI, acute or chronic ischemic heart disease. For example, following an MI, numerous episodes of VT (referred to as phase one episodes) typically occur for several days. Eventually, the phase one VT episodes largely disappear. Several days or weeks later, additional episodes of VT (referred to as phase two episodes) typically begin to occur. Accordingly, in one embodiment, the invention provides methods of treating VT or VF associated with an MI by administering an effective amount of an NGF antagonist to an individual during and/or following an MI. The NGF antagonist can be administered minutes, days, a week or more, and even months following an MI.

In other embodiments, the present invention provides methods for treating cardiac arrhythmia in an individual who has survived an episode of cardiac arrest, an individual who has been resuscitated from near-fatal ventricular fibrillation or ventricular tachycardia, an individual who has suffered at least one episode of myocardial infarction, and/or an individual who has suffered acute or chronic ischemic heart disease, comprising administering an effective amount of an NGF antagonist to the individual.

In another aspect, the invention provides methods of ameliorating, palliating, suppressing or preventing the progression of a cardiac arrhythmia. In another aspect, the invention provides methods for reducing the incidence of a cardiac arrhythmia.

Cardiac arrhythmias are well described in the art. As the skilled practitioner recognizes, symptoms of cardiac arrhythmia can include one or more of: palpitation, chest pain, syncope, lightheadedness, dyspnea, dizziness, vertigo, epilepsy, transient ischemic events, and cardiac arrest.

Diagnosis of cardiac arrhythmia is well known in the art. Cardiac arrhythmia can be detected by any standard diagnostic tool or method, including: clinical examination, electrocardiogram (ECG), a Holter monitor, an echocardiogram (Echo), a standard electrophysiological study (EPS), left ventricular ejection fractions (LVEF), or a treadmill test. It is appreciated that the diagnostics of a person who complains of symptoms that suggest arrhythmia can be inconclusive because of the fleeting nature of arrhythmias.

In another aspect, the invention encompasses methods of delaying development and/or preventing death resulting from cardiac arrhythmia, including preventing SCD. SCD may accompany almost all known heart diseases, such as coronary artery disease, myocardial infarction, mitral insufficiency, hypertrophic cardiomyopathy, and ischemic heart diseases. In most cases, the direct cause of sudden cardiac death is a VT, which triggers VF. As in apparent to those skilled in the art, a cardiac arrhythmia may have preceded death for longer than an hour, days, a week or more, a month, or even a year, yet the individual may have been disposed to death by essentially similar changes in the heart as those in SCD.

Thus, in one aspect, the invention provides methods of preventing SCD associated with (and/or due to) cardiac arrhythmia. In some embodiments, the administered NGF antagonists can prevent the development or progression of VT and/or VF, and may prevent or reduce the risk of SCD. In other embodiments, the invention provides for administration of an NGF antagonist before or during a first episode of ventricular tachycardia; before or during an episode of ventricular tachycardia; or before or during ventricular fibrillation. In still other embodiments, the invention provides methods of preventing SCD in an individual who has had a previous Ml, and is thus at increased or high risk of SCD. The risk of SCD is even greater if the individual also has atriventricular (AV) block, i.e., there is a partial or total interruption of the conduction of electrical impulses from the atria to the ventricles. Accordingly, one embodiment of the present invention encompasses methods of preventing SCD by administering an NGF antagonist following the occurrence of an MI, for example, minutes, hours, days, a week or more, weeks, or even months after the occurrence of an MI. In some embodiments, the individual has a phase one VT or a phase two VT.

In another aspect, the invention provides methods for enhancing cardiac function in individuals in need thereof (such as an individual with MI) comprising administering an effective amount of an NGF antagonist. In some embodiments, the individual has one or more symptoms of cardiac arrhythmia In other embodiments, the individual is at risk of arrhythmia. In still other embodiments, the individual is an individual who has survived an episode of cardiac arrest, or an individual who has been resuscitated from near-fatal ventricular fibrillation.

Methods for Treating Individuals at Risk of an NGF Associated Cardiac Arrhythmia

The present invention can also be used before the first episode of cardiac arrhythmia or cardiac arrest (including SCD). That is, any individual that is at risk of developing NGF-associated cardiac arrhythmia can be treated.

Methods for determining risk of developing a cardiac arrhythmia are well-known in the art. For example, electrophysiological studies (EPS) using programmed ventricular stimulation can be used to identify individuals who are at risk of cardiac arrhythmia. Methods such as measurement of left ventricular ejection fraction (LVEF), measurement of heart rate variability, baroreflex responses, signal-averaged electrocardiography (SAECG), ambient arrhythmia, and QT dispersion, can also be used, either alone or in different combinations, to identify individuals who are at risk of cardiac arrhythmia. T wave alternans (TWA), a heart-rate-dependent measure of arrhythmia vulnerability, can also be used to predict inducibility of ventricular tachycardia with programmed stimulation and to predict spontaneous arrhythmic events. See, e.g., Osman A F, et al., Current Opinion in Cardiology 17:1-5 (2002). Risk can also be indicated by electrocardiographic changes, which include, but are not limited to, ST-segment depression, T-wave inversion, and prolonged QT interval. Risk can also be indicated by genetic analysis, including as described below.

An individual who displays one or more risk factors that correlate with cardiac arrhythmia is considered to be an individual at a risk of developing a cardiac arrhythmia. Preferably, the individual displays more than one of the risk factors, more preferably more than two of the risk factors. High (i.e., increased) risk may be indicated, for example, on the basis of the presence of precursor diseases, such as myocardial infarction, acute or chronic ischemic heart disease, cardiomyopathy, congestive heart disease, dysrhythmia, hypertensive heart disease, left ventricular hypertrophy, coronary heart disease, and angina. Furthermore, risk factors that predispose an individual to the above-described heart diseases are also risk factors for cardiac arrhythmia. These factors include, but are not limited to, heart rate variability, elevated heart rate, reduced vagal activity, nonsustained VT, ventricular premature beats, elevated LDL-cholesterol, smoking, diabetes mellitus, male gender, and old age.

Risk of cardiac arrhythmia may also be indicated on the basis of inherited genetic abnormalities, such as long QT interval, Brugada syndrome, hypertrophic cardiomyopathy, arrhythmogenic right ventricular cardiomyopathy, and catacholaminergic polymorphic ventricular tachycardia. Furthermore, family history of heart diseases, medication history, and/or a history of exposure to an environmental substance (such as cigarette smoke) which is known or suspected of being able to increase the risk of heart diseases may also be among the risk factors.

Because all risk factors for developing cardiac arrhythmia are not known, and the interplay among these factors (in terms of overall risk) are not fully understood, it is clear to one skilled in the art that individuals suitable for administration of an NGF antagonist for the purposes of this invention can have clinical features in common, and that individuals not falling clearly in the categories described above can nonetheless be considered suitable candidates for administration of an NGF antagonist. A skilled clinician can make an empirical determination whether an individual is suitable for NGF antagonist treatment. For example, an individual with a familial (i.e., genetic) history of heart disease could be considered “at risk”, even though no previous disease in this individual has been observed. In this context, administration of an NGF antagonist to such an individual could result in delay of occurrence of disease, to the extent that the individual does not develop the disease within his or her lifetime (or develops it later than would have been expected). Another example is an individual who is being treated using traditional modes of therapy, and who is showing clinical responsiveness to the therapy (remission). Such an individual may be adjudged as “at risk”, even though the initial course of therapy is not yet completed, due to projection of clinical progress by the clinician, and can be a suitable candidate for receiving an NGF antagonist before completion of the initial therapy. The clinician, as one skilled in the art, has discretion to determine whether treatment using an NGF antagonist should be adopted.

It is also evident that administration of an NGF antagonist may be indicated even if an individual is not adjudged to be at risk (i.e., is “low risk”) according to concurrent clinical risk assessment criteria. For instance, an individual who has been successfully treated and is not considered at risk (due, for example, to the lack of detectable disease at the time of diagnosis) may nonetheless be a candidate for receiving an NGF antagonist as a precautionary measure. Thus, the risk of disease progression may be lowered even further by administration of an NGF antagonist. As another example, an individual may believe that he or she is at risk of disease development, and may decide that receiving NGF antagonist would reduce this risk.

Thus, the present invention encompasses methods of treating cardiac arrhythmia in an individual at risk of developing a cardiac arrhythmia comprising administering an effective amount of an NGF antagonist. In other embodiments, the present invention encompasses administering an NGF antagonist to individuals at risk of developing a cardiac arrhythmia, to prevent or delay the development of symptoms of a cardiac arrhythmia. The present invention also encompasses methods of enhancing cardiac function and/or decreasing sympathetic innervation or sympathetic activity comprising administering an NGF antagonist to individuals at risk of developing a cardiac arrhythmia.

The invention also encompasses methods of reducing risk of development or progression of a cardiac arrhythmia in an individual at risk of developing cardiac arrhythmia. In these methods, an effective amount of an NGF antagonist is administered to an individual at risk for development or progression of a cardiac arrhythmia. “Reducing risk of development or progression” means that the risk of development and/or progression of a cardiac arrhythmia is lower in individuals receiving an NGF antagonist than those individuals (having the same risk of cardiac arrhythmia) who do not. In some embodiments, the invention provides methods of reducing risk of development or progression in an individual at low risk of developing cardiac arrhythmia.

The invention also encompasses methods of reducing risk of occurrence of death (such as SCD) due to a cardiac arrhythmia in an individual at risk of developing cardiac arrhythmia. In these methods, an effective amount of an NGF antagonist is administered to an individual at risk of occurrence of death due to the cardiac arrhythmia. “Reducing risk of occurrence of death” means that the risk of death is lower in individuals receiving an NGF antagonist than those individuals (having the same risk of death) who do not. The present invention encompasses methods of reducing risk of both SCD and death resulting from a long-lasting arrhythmia.

Methods of Suppressing Sympathetic Hyperinnervation

The present invention also encompasses methods comprising administering an NGF antagonist to an individual to suppress NGF-induced cardiac sympathetic hyperinnervation. For individuals having a symptom of cardiac arrhythmia and thus already manifesting undesired sympathetic hyperinnervation, inhibiting NGF activity serves to reverse the sympathetic hyperinnervation and/or hyperactivity, and/or reduce or prevent the progression of the sympathetic hyperinnervation and/or hyperactivity. Alternatively, the NGF antagonist can be administered to an individual at risk of developing cardiac arrhythmia to reduce or prevent the progression of sympathetic hyperinnervation. In cases where sympathetic hyperinnervation has not yet occurred, the NGF antagonist serves to prevent or delay the development of sympathetic hyperinnervation (such as cardiac sympathetic hyperinnervation). It is understood that “sympathetic hyperinnervation” and “sympathetic hyperactivity” encompass localized hyperinnervation and/or hyperactivity as well as generalized hyperinnervation and/or hyperactivity (as well as intermediate levels of hyperinnervation and/or hyperactivity).

Sympathetic hyperinnervation and hyperactivity can be evaluated by various methods known in the art. For example, regional sympathetic activities can be studied in an individual using electrophysiological methods measuring sympathetic nerve firing and neurochemical techniques providing quantitation of nonadrenaline spill over to plasma from sympathetic nerves to individual organs. Kay, J. Cardiovascular Pharmacol. 35 (7 Suppl. 4):S1-7 (2000). Power spectrum analysis can also be performed to determine the contribution of sympathetic nerve activity to fluctuations in cardiac rhythm. Furthermore, radiolabeled analogs of norepinephrine, such as ¹²³I-metaiodobenzylguanidine (MIBG), can be actively taken up by the sympathetic nerve terminals of the heart and thereby permit direct regional assessment of cardiac sympathetic integrity, density and regularity through scintigraphic imaging. See Carrio, J. Nuc. Med., 42(7):1062-1076; Estorch M, J. Nucl. Med. 40(6):911-6; Stanton et al., J. Am. Coll. Cardiol. 14:1519-1526 (1989); Minardo et al., Circulation 78:1008-1019 (1988).

NGF Antagonists

The methods of the invention use an NGF antagonist, which refers to any molecule that blocks, suppresses or reduces (including significantly reduces) NGF biological activity, including downstream pathways mediated by NGF signaling, such as receptor binding and/or elicitation of a cellular response to NGF. The term “antagonist” implies no specific mechanism of biological action whatsoever, and is deemed to expressly include and encompass all possible pharmacological, physiological, and biochemical interactions with NGF and its consequences which can be achieved by a variety of different, and chemically divergent, compositions. Exemplary NGF antagonists include, but are not limited to, an anti-NGF antibody, an anti-sense molecule directed to an NGF (including an anti-sense molecule directed to a nucleic acid encoding NGF), an anti-sense molecule directed to an NGF receptor (such as TrkA and/or p75) (in some embodiments, an anti-sense molecule directed to a nucleic acid encoding a NGF receptor), an NGF inhibitory compound, an NGF structural analog, a dominant-negative mutation of a TrkA receptor that binds an NGF, a TrkA immunoadhesin, an anti-TrkA antibody, an anti-p75 antibody, and a kinase inhibitor. For purpose of the present invention, it will be explicitly understood that the term “antagonist” encompasses all the previously identified terms, titles, and functional states and characteristics whereby the NGF itself, an NGF biological activity (including but not limited to its ability to ability to stimulate sympathetic hyperinnervation), or the consequences of the biological activity, are substantially nullified, decreased, or neutralized in any meaningful degree. In some embodiments, an NGF antagonist (e.g., an antibody) binds (physically interacts with) NGF, binds to an NGF receptor (such as trkA receptor or p75 receptor), and/or reduces (impedes and/or blocks) downstream NGF receptor signaling.

Anti-NGF Antibodies

In some embodiments of the invention, the NGF antagonist comprises an anti-NGF antibody. An anti-NGF antibody should exhibit any one or more of the following characteristics: (a) bind to NGF; (b) inhibit NGF biological activity or downstream pathways mediated by NGF signaling function; (c) inhibit sympathetic hyperinnervation or hyperactivity; (d) treat or prevent development of NGF-associated cardiac arrhythmia; (e) block or decrease NGF receptor activation (including trkA receptor dimerization and/or autophosphorylation); (f) increase clearance of NGF; (g) inhibit (reduce) NGF synthesis, production or release; (h) enhance cardiac function.

Anti-NGF antibodies are known in the art, see, e.g., WO 01/78698, WO 01/64247, U.S. Pat. Nos. 5,844,092, 5,877,016, and 6,153,189; Hongo et al., Hybridoma 19:215-227 (2000); Cell. Molec. Biol. 13:559-568 (1993); GenBank Accession Nos. U39608, U39609, L17078, or L17077.

In some embodiments, the anti-NGF antibody is a humanized mouse anti-NGF monoclonal antibody termed antibody “E3”, which comprises the human heavy chain IgG2a constant region containing the following mutations: A330P331 to S330S331 (amino acid numbering with reference to the wildtype IgG2a sequence; see Eur. J. Immunol. (1999) 29:2613-2624); the human light chain kappa constant region; and the heavy and light chain variable regions shown in Tables 1 and 2. The binding affinity of antibody E3 to human NGF is about 0.1 nM.

TABLE 1
Heavy chain variable region

QVQLQESGPGLVKPSETLSLTCTVSGFSLIGYDLNWIRQPPGKGLEWIGI
IWGDGTTDYNSAVKSRVTISKDTSKNQFSLKLSSVTAADTAVYYCARGGY
WYATSYYFDYWGQGTLVTVS. (SEQ ID NO:1)

TABLE 2
Light chain variable region

DIQMTQSPSSLSASVGDRVTITCRASQSISNNLNWYQQKPGKAPKLLIYY
TSRFHSGVPSRFSGSGSGTDFTFTISSLQPEDIATYYCQQEHTLPYTFGQ
GTKLEIKRT. (SEQ ID NO:2)

The following polynucleotides encoding the heavy chain variable region or the light chain variable region were deposited at the ATCC on Jan. 8, 2003:

| | | ATCC | Date of |
|---|---|---|---|
| Material | | Accession No. | Deposit |
| Vector Eb.911.3E | E3 light chain V | PTA-4893 | Jan. 8, 2003 |
| | region | | |
| Vector Eb.pur.911.3E | E3 light chain V | PTA-4894 | Jan. 8, 2003 |
| | region | | |
| Vector Db.911.3E | E3 heavy chain V | PTA-4895 | Jan. 8, 2003 |
| | region | | |

Vector Eb.911.3E is a polynucleotide encoding the light chain variable region shown in Table 2; vector Eb.pur.911.3E is a polynucleotide encoding the light chain variable region shown in Table 2 and vector Db.911.3E is a polynucleotide encoding the heavy chain variable region shown in Table 1.

In another embodiment, the anti-NGF antibody comprises one or more CDR(s) of antibody E3 (such as one, two, three, four, five, or, in some embodiments, all six CDRs from E3). Determination of CDR regions is well within the skill of the art.

The antibodies useful in the present invention can encompass monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric and humanized antibodies). For purposes of this invention, the antibody reacts with NGF in a manner that inhibits NGF and/or downstream pathways mediated by the NGF signaling function. In one embodiment, the antibody is a human antibody which recognizes one or more epitopes on human NGF. In another embodiment, the antibody is a mouse or rat antibody which recognizes one or more epitopes on human NGF. In another embodiment, the antibody recognizes one or more epitopes on an NGF selected from the group consisting of: primate, canine, feline, equine, and bovine. In other embodiments, the antibody comprises a modified constant region, such as a constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT/GB99/01441; and/or UK patent application No. 9809951.8.

The binding affinity of an anti-NGF antibody to NGF (such as hNGF) can be about 0.10 to about 0. 80 nM, about 0.15 to about 0.75 nM and about 0.18 to about 0.72 nM. In one embodiment, the binding affinity is between about 2 pM and 22 pM. In some embodiment, the binding affinity is about 10 nM. In other embodiments, the binding affinity is less than about 10 nM. In other embodiments, the binding affinity is about 0.1 nM. In other embodiments, the binding affinity is less than about 0.1 nM. In other embodiments, the binding affinity is any of about 100 nM, about 50 nM, about 10 nM, about 1 nM, about 500 pM, about 100 pM, or about 50 pM to any of about 2 pM, about 5 pM, about 10 pM, about 15 pM, about 20 pM, or about 40 pM. In some embodiments, the binding affinity is any of about 100 nM, about 50 nM, about 10 nM, about 1 nM, about 500 pM, about 100 pM, or about 50 pM, or less than about 50 pM. In some embodiments, the binding affinity is less than any of about 100 nM, about 50 nM, about 10 nM, about 1 nM, about 500 pM, about 100 pM, or about 50 pM. In still other embodiments, the binding affinity is about 2 pM, about 5 pM, about 10 pM, about 15 pM, about 20 pM, about 40 pM, or greater than about 40 pM.

One way of determining binding affinity of antibodies to NGF is by measuring affinity of monofunctional Fab fragments of the antibody. To obtain monofunctional Fab fragments, an antibody (for example, IgG) can be cleaved with papain or expressed recombinantly. The affinity of an anti-NGF Fab fragment of an antibody can be determined by surface plasmon resonance (BlAcore3000™ surface plasmon resonance (SPR) system, BIAcore, INC, Piscaway N.J.). CM5 chips can be activated with N-ethyl-N′-(3-dimethylaminopropyl)-carbodiinide hydrochloride (EDC) and N-hydroxysuccinimide (NHS) according to the supplier's instructions. Human NGF can be diluted into 10 mM sodium acetate pH 4.0 and injected over the activated chip at a concentration of 0.005 mg/mL. Using variable flow time across the individual chip channels, two ranges of antigen density can be achieved: 100-200 response units (RU) for detailed kinetic studies and 500-600 RU for screening assays. The chip can be blocked with ethanolamine. Regeneration studies have shown that a mixture of Pierce elution buffer (Product No. 21004, Pierce Biotechnology, Rockford, Ill.) and 4 M NaCl (2:1) effectively removes the bound Fab while keeping the activity of hNGF on the chip for over 200 injections. HBS-EP buffer (0.01M HEPES, pH 7.4, 0.15 NaCl, 3 mM EDTA, 0.005% Surfactant P29) is used as running buffer for the BIAcore assays. Serial dilutions (0.1-10× estimated K_{D}) of purified Fab samples are injected for 1 min at 100 μL/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values are calculated as k_{off}/kₒₙ.

In some embodiments, the antibody binds human NGF, and does not significantly bind an NGF from another vertebrate species (in some embodiments, mammalian). In some embodiments, the antibody binds human NGF as well as one or more NGF from another vertebrate species (in some embodiments, mammalian). In still other embodiments, the antibody binds NGF and does not significantly cross-react with other neurotrophins (such as the related neurotrophins, NT3, NT4/5, and/or BDNF). In some embodiments, the antibody binds NGF as well as at least one other neurotrophin. In some embodiments, the antibody binds to a mammalian species of NGF, such as horse or dog, but does not significantly bind to NGF from another mammalian species.

The epitope(s) can be continuous or discontinuous. In one embodiment, the antibody binds essentially the same hNGF epitopes as an antibody selected from one or more of the following: MAb 911, MAb 912, and MAb 938 as described in Hongo et al., Hybridoma 19:215-227 (2000). In another embodiment, the antibody binds essentially the same hNGF epitope as MAb 911. In still another embodiment, the antibody binds essentially the same epitope as MAb 909. Hongo et al., supra. For example, the epitope may comprise one or more of: residues K32, K34 and E35 within variable region 1 (amino acids 23-35) of hNGF; residues F79 and T81 within variable region 4 (amino acids 81-88) of hNGF; residues H84 and K88 within variable region 4; residue R103 between variable region 5 (amino acids 94-98) of hNGF and the C-terminus (amino acids 111-118) of hNGF; residue E11 within pre-variable region 1 (amino acids 10-23) of hNGF; Y52 between variable region 2 (amino acids 40-49) of hNGF and variable region 3 (amino acids 59-66) of hNGF; residues L112 and S113 within the C-terminus of hNGF; residues R59 and R69 within variable region 3 of hNGF; or residues V18, V20, and G23 within pre-variable region 1 of hNGF. In addition, an epitope can comprise one or more of the variable region 1, variable region 3, variable region 4, variable region 5, the N-terminus region, and/or the C-terminus of hNGF. In still another embodiment, the antibody significantly reduces the solvent accessibility of residue R103 of hNGF. It is understood that although the epitopes described above relate to human NGF, one of ordinary skill can align the structures of human NGF with the NGF of other species and identify likely counterparts to these epitopes.

In one aspect, antibodies (e.g., human, humanized, mouse, chimeric) that can inhibit NGF may be made by using immunogens that express full length or partial sequence of NGF. In another aspect, an immunogen comprising a cell that overexpresses NGF may be used. Another example of an immunogen that can be used is NGF protein that contains full-length NGF or a portion of the NGF protein.

The anti-NGF antibodies may be made by any method known in the art. The route and schedule of immunization of the host animal are generally in keeping with established and conventional techniques for antibody stimulation and production, as further described herein. General techniques for production of human and mouse antibodies are known in the art and are described herein.

It is contemplated that any mammalian subject including humans or antibody producing cells therefrom can be manipulated to serve as the basis for production of mammalian, including human, hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-NGF monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for NGF, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with a human NGF, or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaradehyde, succinic anhydride, SOCl2, or R1N═C═NR, where R and R1 are different alkyl groups, can yield a population of antibodies (e.g., monoclonal antibodies).

If desired, the anti-NGF antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to NGF and greater efficacy in inhibiting NGF. It will be apparent to one of skill in the art that one or more polynucleotide changes can be made to the anti-NGF antibody and still maintain its binding ability to NGF.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules which limits the duration and effectiveness of therapeutic applications of those moieties in human recipients. Other methods of humanizing antibodies that may also be utilized are disclosed by Daugherty et al., Nucl. Acids Res. 19:2471-2476 (1991) and in U.S. Pat. Nos. 6,180,377; 6,054,297; 5,997,867; and 5,866,692; 6,210,671; 6,350,861; and PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743; 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin.

It is apparent that although the above discussion pertains to humanized antibodies, the general principles discussed are applicable to customizing antibodies for use, for example, in dogs, cats, primate, equines and bovines. It is further evident that one or more of aspects of humanizing antibodies described herein may be combined, e.g., CDR grafting, framework mutation and CDR mutation.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. Vaccine 19:2756 (2001); Lonberg, N. and D. Huszar Int. Rev. Immunol 13:65 (1995); and Pollock, et al., J Immunol Methods 231:147(1999). Methods for making derivatives of antibodies, e.g., humanized, single chain, etc. are known in the art.

Immunoassays and flow cytometry sorting techniques such as fluorescence activated cell sorting (FACS) can also be employed to isolate antibodies that are specific for NGF.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation. In some embodiments, the carrier comprises a moiety that targets the myocardium.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors (such as expression vectors disclosed in WO87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., WO87/04462. The DNA also may be modified, for example, by substituting the coding sequence for human heavy and light chain constant domains in place of the homologous murine sequences, Morrison et al., Proc. Nat. Acad. Sci. 81:6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-NGF monoclonal antibody herein.

Anti-NGF antibodies may be characterized using methods well known in the art. For example, one method is to identify the epitope to which it binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an anti-NGF antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an anti-NGF antibody. In another example, the epitope to which the anti-NGF antibody binds can be determined in a systematic screening by using overlapping peptides derived from the NGF sequence and determining binding by the anti-NGF antibody. According to the gene fragment expression assays, the open reading frame encoding NGF is fragmented either randomly or by specific genetic constructions and the reactivity of the expressed fragments of NGF with the antibody to be tested is determined. The gene fragments may, for example, be produced by PCR and then transcribed and translated into protein in vitro, in the presence of radioactive amino acids. The binding of the antibody to the radioactively labeled NGF fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant NGF in which various fragments of the NGF polypeptide have been replaced (swapped) with sequences from a closely related, but antigenically distinct protein (such as another member of the neurotrophin protein family). By assessing binding of the antibody to the mutant NGF, the importance of the particular NGF fragment to antibody binding can be assessed.

Yet another method which can be used to characterize an anti-NGF antibody is to use competition assays with other antibodies known to bind to the same antigen, i.e., various fragments on NGF, to determine if the anti-NGF antibody binds to the same epitope as other antibodies. Competition assays are well known to those of skill in the art. Examples of antibodies that can be used in the competition assays include MAbs 911, 912, and/or 938, as described in Hongo et al., Hybridoma 19:215-227 (2000).

Other NGF Antagonists

NGF antagonists other than anti-NGF antibodies may be used. In some embodiments of the invention, the NGF antagonist comprises at least one antisense molecule capable of blocking or decreasing the expression of a functional NGF. Nucleotide sequences of the NGF are known and are readily available from publicly available databases. See, e.g., Borsani et al., Nuc. Acids Res. 1990, 18, 4020; Accession Number NM 002506; Ullrich et al., Nature 303:821-825 (1983). It is routine to prepare antisense oligonucleotide molecules that will specifically bind NGF mRNA without cross-reacting with other polynucleotides. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifications such as, for example, phosphorothioate linkages, and 2′-O sugar modifications well know in the art. Exemplary antisense molecules include the NGF antisense molecules described in U.S. Publication No. 20010046959; see also http://www.rna-tec.com/repair.htm.

Alternatively, NGF expression and/or release (and/or NGF receptor expression) can be decreased using gene knockdown, morpholino oligonucleotides, RNAi, or ribozymes, methods that are well-known in the art. See http://www.macalester.edu/˜montgomery/RNAi.html; http://pub32.ezboard.com/fmorpholinosfrm19.showMessage?topicID=6.topic; http://www.highveld.com/ribozyme.html.

In other embodiments, the NGF antagonist comprises at least one NGF inhibitory compound. As used herein, “NGF inhibitory compound” refers to a compound other than an anti-NGF antibody that directly or indirectly reduces, inhibits, neutralizes, or abolishes NGF biological activity. An NGF inhibitory compound should exhibit any one or more of the following characteristics: (a) bind to NGF; (b) inhibit NGF biological activity or downstream pathways mediated by NGF signaling function; (c) inhibit or reduce sympathetic hyperinnervation or hyperactivity; (d) treat or prevent development of NGF-associated cardiac arrhythmia; (e) block or decrease NGF receptor activation (including trkA receptor dimerization and/or autophosphorylation); (f) increase clearance of NGF; (g) inhibit (reduce) NGF synthesis, production or release; (h) enhance cardiac function. Exemplary NGF inhibitory compounds include the small molecule NGF inhibitors described in U.S. Publication No. 20010046959; the compounds that inhibit NGF's binding to p75, as described in PCT Publication No. WO 00/69829; the compounds that inhibit NGF's binding to TrkA and/or p75, as described in PCT Publication No. WO 98/17278. Additional examples of NGF inhibitory compounds include the compounds described in WO 02/17914, WO 02/20479, U.S. Pat. Nos. 5,342,942, 6,127,401, and 6,359,130. Further exemplary NGF inhibitory compounds are compounds that are competitive inhibitors of NGF. See U.S. Pat. No. 6,291,247. Furthermore, one skilled in the art can prepare other NGF inhibitory compounds, including other small molecule NGF inhibitory compounds.

In some embodiments, a NGF inhibitory compound binds NGF. Exemplary sites of targeting (binding) include, but are not limited to, the portion(s) of the NGF that binds to the TrkA receptor and/or p75 receptor, and those portions of the NGF that are adjacent to the receptor-binding region and which are responsible, in part, for the correct three-dimensional shape of the receptor-binding portion. In another embodiment, a NGF inhibitory compound binds an NGF receptor (such as trkA and/or p75) and inhibits a NGF biological activity. Exemplary sites of targeting include those portions of trkA and/or p75 that bind to NGF.

In embodiments comprising small molecule NGF inhibitory compounds, the small molecules can have a molecular weight of about any of 100 to 20,000 daltons, 500 to 15,000 daltons, or 1000 to 10,000 daltons. Libraries of small molecules are commercially available. The small molecules can be administered using any means known in the art, including inhalation; intraperitoneally, intravenously, intramuscularly, subcutaneously, intrathecally, intraventricularly, orally, enterally, parenterally, intranasally, or dermally. In general, when the NGF antagonist according to the invention is a small molecule, it will be administered at the rate of 0.1 to 300 mg/kg of the weight of the patient divided into one to three or more doses. In some embodiments, for an adult patient of normal weight, doses ranging from 1 mg to 5g per dose may be administered.

In other embodiments, the NGF antagonist comprises at least one NGF structural analog. “NGF structural analogs” in the present invention refer to compounds that have a similar 3-dimensional structure as part of that of NGF and which bind to an NGF receptor under physiological conditions in vitro or in vivo, wherein the binding at least partially inhibits an NGF biological activity. In one embodiment, the NGF structural analog binds to a TrkA and/or a p75 receptor. In one embodiment, the NGF structural analog inhibits NGF's activity to promote sympathetic hyperinnervation. Exemplary NGF structural analogs include, but are not limited to, the bicyclic peptides described in PCT Publication No. WO 97/15593; the bicyclic peptides described in U.S. Pat. No. 6,291,247; the cyclic compounds described in U.S. Pat. No. 6,017,878; and NGF-derived peptides described in PCT Publication No. WO 89/09225. Suitable NGF structural analogs can also be designed and synthesized through molecular modeling of NGF-receptor binding, for example by the method described in WO 98/06048. The NGF structural analogs can be monomers or dimers/oligomers in any desired combination of the same or different structures to obtain improved affinities and biological effects.

In other embodiments, the invention provides an NGF antagonist comprising at least one dominant-negative mutant of the TrkA receptor or p75 receptor. One skilled in the art can prepare dominant-negative mutants of the TrkA receptor such that the receptor will bind the NGF and, thus, act as a “sink” to capture NGFs. The dominant-negative mutants, however, will not have the normal bioactivity of the TrkA receptor upon binding to NGF. Exemplary dominant-negative mutants include, but are not limited to, the mutants described in the following references: Li et al., Proc. Natl. Acad. Sci. USA 1998, 95, 10884; Eide et al., J. Neurosci. 1996, 16, 3123; Liu et al., J. Neurosci 1997, 17, 8749; Klein et al., Cell 1990, 61, 647; Valenzuela et al., Neuron 1993, 10, 963; Tsoulfas et al., Neuron 1993, 10, 975; and Lamballe et al., EMBO J. 1993, 12, 3083, each of which is incorporated herein by reference in its entirety. The dominant negative mutants can be administered in protein form or in the form of an expression vector such that the dominant negative mutant (such as a mutant TrkA receptor) is expressed in vivo. The protein or expression vector can be administered using any means known in the art, including intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. In another embodiment, the protein or expression vector is administered directly to the sympathetic trunk or ganglion, or into a coronary artery, atrium, ventrical, or pericardium. One skilled in the art is familiar with administration of expression vectors to obtain expression of an exogenous protein in vivo. See, e.g., U.S. Pat. Nos. 6,436,908; 6,413,942; 6,376,471.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA can be used during a gene therapy protocol. The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; U.S. Pat. No. 5, 219,740; PCT Publication Nos. WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581.

It is also apparent that an expression vector can be used to direct expression of any of the protein-based NGF antagonists described herein (e.g., NGF antibody, TrkA immunoadhesin, etc.). For example, TrkA receptor fragments that are capable of blocking (from partial to complete blocking) NGF and/or a NGF biological activity are known in the art.

In another embodiment, the NGF antagonist comprises at least one TrkA immunoadhesin. TrkA immunoadhesins as used herein refer to soluble chimeric molecules comprising the extracellular domain of a TrkA receptor and an immunoglobulin sequence, which retains the binding specificity of the TrkA receptor and is capable of binding to NGF and blocking the biological activity of NGF.

TrkA immunoadhesins are known in the art, and have been found to block the binding of NGF to the TrkA receptor, and thereby inhibit an NGF biological activity. See, e.g., U.S. Pat. No. 6,153,189. In one embodiment, the TrkA immunoadhesin comprises a fusion of a TrkA receptor amino acid sequence (or an amino acid sequence that substantially retains the binding specificity of the trkA receptor) capable of binding NGF and an immunoglobulin sequence. In some embodiments, the TrkA receptor is a human TrkA receptor sequence, and the fusion is with an immunoglobulin constant domain sequence. In other embodiments, the immunoglobulin constant domain sequence is an immunoglobulin heavy chain constant domain sequence. In other embodiments, the association of two TrkA receptor-immunoglobulin heavy chain fusions (e.g., via covalent linkage by disulfide bond(s)) results in a homodimeric immunoglobulin-like structure. An immunoglobulin light chain can further be associated with one or both of the TrkA receptor-immunoglobulin chimeras in the disulfide-bonded dimer to yield a homotrimeric or homotetrameric structure. Examples of suitable TrkA immunoadhesins include those described in U.S. Pat. No. 6,153,189.

In another embodiment, the NGF antagonist comprises at least one anti-TrkA antibody capable of blocking, suppressing, altering, and/or reducing NGF physical interaction with the TrkA receptor and/or downstream signaling, whereby an NGF biological activity is reduced and/or blocked. Anti-TrkA antibodies are known in the art. Exemplary anti-TrkA antibodies include those described in PCT Publication Nos. WO 97/21732, WO 00/73344, WO 02/15924, and U.S. Publication No. 20010046959. In another embodiment, the NGF antagonist comprises at least one anti-p75 antibody capable of blocking, suppressing and/or reducing NGF physical interaction with the p75 receptor and/or downstream signaling, whereby an NGF biological activity is reduced and/or blocked.

In another embodiment, the NGF antagonist comprises at least one kinase inhibitor capable of inhibiting downstream kinase signaling associated with trkA and/or p75 receptor activity. An exemplary kinase inhibitor is K252a or K225b, which is known in the art and described in Knusel et al., J. Neurochem. 59:715-722 (1992); Knuet al., J. Neurochemistry 57:955-962 (1991); Koizumi et al., J. Neuroscience 8:715-721 (1988); Hirata et al., Chemical Abstracts 111 :728, XP00204135, see abstract and 12th Collective Chemical Substance Index, p. 34237, c. 3 (5-7), 55-60, 66-69), p. 34238, c.1 (41-44), c.2 (25-27, 32-33), p. 3423, c.3 (48-50, 52-53); U.S. Pat. No. 6,306,849.

It is expected that a number of other categories of NGF antagonists will be identified if sought for by the clinician.

Identification of NGF Antagonists

Anti-NGF antibodies and other NGF antagonists can be identified or characterized using methods known in the art, whereby reduction, amelioration, or neutralization of an NGF biological activity is detected and/or measured. For example, a kinase receptor activation (KIRA) assay described in U.S. Pat. Nos. 5,766,863 and 5,891,650, can be used to identify NGF antagonists. This ELISA-type assay is suitable for qualitative or quantitative measurement of kinase activation by measuring the autophosphorylation of the kinase domain of a receptor protein tyrosine kinase (hereinafter “rPTK”), e.g. TrkA receptor, as well as for identification and characterization of potential antagonists of a selected rPTK, e.g., TrkA. The first stage of the assay involves phosphorylation of the kinase domain of a kinase receptor, for example, a TrkA receptor, wherein the receptor is present in the cell membrane of a eukaryotic cell. The receptor may be an endogenous receptor or nucleic acid encoding the receptor, or a receptor construct, may be transformed into the cell. Typically, a first solid phase (e.g., a well of a first assay plate) is coated with a substantially homogeneous population of such calls (usually a mammalian call line) so that the cells adhere to the solid phase. Often, the cells are adherent and thereby adhere naturally to the first solid phase. If a “receptor construct” is used, it usually comprises a fusion of a kinase receptor and a flag polypeptide. The flag polypeptide is recognized by the capture agent, often a capture antibody, in the ELISA part of the assay. An analyte, such as a candidate anti-NGF antibody or other NGF antagonist, is then added together with NGF to the wells having the adherent cells, such that the tyrosine kinase receptor (e.g. TrkA receptor) is exposed to (or contacted with) NGF and the analyte. This assay enables identification of antibodies (or other NGF antagonists) that inhibit activation of TrkA by its ligand NGF. Following exposure to NGF and the analyte, the adhering cells are solubilized using a lysis buffer (which has a solubilizing detergent therein) and gentle agitation, thereby releasing cell lysate which can be subjected to the ELISA part of the assay directly, without the need for concentration or clarification of the cell lysate.

The cell lysate thus prepared is then ready to be subjected to the ELISA stage of the assay. As a first step in the ELISA stage, a second solid phase (usually a well of an ELISA microtiter plate) is coated with a capture agent (often a capture antibody) which binds specifically to the tyrosine kinase receptor, or, in the case of a receptor construct, to the flag polypeptide. Coating of the second solid phase is carried out so that the capture agent adheres to the second solid phase. The capture agent is generally a monoclonal antibody, but, as is described in the examples herein, polyclonal antibodies may also be used. The cell lysate obtained is then exposed to, or contacted with, the adhering capture agent so that the receptor or receptor construct adheres to (or is captured in) the second solid phase. A washing step is then carried out, so as to remove unbound cell lysate, leaving the captured receptor or receptor construct. The adhering or captured receptor or receptor construct is then exposed to, or contacted with, an anti-phosphotyrosine antibody which identifies phosphorylated tyrosine residues in the tyrosine kinase receptor. In some embodiments, the anti-phosphotyrosine antibody is conjugated (directly or indirectly) to an enzyme which catalyses a color change of a non-radioactive color reagent. Accordingly, phosphorylation of the receptor can be measured by a subsequent color change of the reagent. The enzyme can be bound to the anti-phosphotyrosine antibody directly, or a conjugating molecule (e.g., biotin) can be conjugated to the anti-phosphotyrosine antibody and the enzyme can be subsequently bound to the anti-phosphotyrosine antibody via the conjugating molecule. Finally, binding of the anti-phosphotyrosine antibody to the captured receptor or receptor construct is measured, e.g., by a color change in the color reagent

An NGF antagonist can also be identified by incubating a candidate agent with NGF and monitoring any one or more of the following characteristics: (a) binding to NGF; (b) inhibiting NGF biological activity or downstream pathways mediated by NGF signaling function; (c) inhibiting sympathetic hyperinnervation or hyperactivity; (d) treating or preventing development of NGF-associated cardiac arrhythmia; (e) blocking or decreasing NGF receptor activation (including trkA dimerization and/or autophosphorylation); (f) increasing clearance of NGF; (g) enhancing cardiac function; (h) inhibiting (reducing) NGF synthesis, production or release. In some embodiments, an NGF antagonist is identified by incubating an candidate agent with NGF and monitoring binding and attendant reduction or neutralization of a biological activity of NGF. The binding assay may be performed with purified NGF polypeptide(s), or with cells naturally expressing, or transfected to express, NGF polypeptide(s). In one embodiment, the binding assay is a competitive binding assay, where the ability of a candidate antibody to compete with a known NGF antagonist (such as an anti-NGF antibody) for NGF binding is evaluated. The assay may be performed in various formats, including the ELISA format. In other embodiments, an NGF antagonist is identified by incubating a candidate agent with NGF and monitoring attendant inhibition of trkA receptor dimerization and/or autophosphorylation.

Following initial identification, the activity of a candidate NGF antagonist can be further confirmed and refined by bioassays, known to test the targeted biological activities. Alternatively, bioassays can be used to screen candidates directly. For example, NGF promotes a number of morphologically recognizable changes in responsive cells. These include, but are not limited to, promoting the differentiation of PC12 cells and enhancing the growth of neurites from these cells (Urfer et al., Biochem. 36: 4775-4781 (1997); Tsoulfas et al., Neuron 10: 975-990 (1993)), promoting neurite outgrowth from explants of responsive sensory and sympathetic ganglia (Levi-Montalcini, R. and Angeletti, P. Nerve growth factor. Physiol. Rev. 48, 534-569, 1968) and promoting the survival of NGF dependent neurons such as embryonic dorsal root ganglion, trigeminal ganglion, or sympathetic ganglion neurons (e.g., Chun & Patterson, Dev. Biol. 75:705-711, (1977); Buchman & Davies, Development 118:989-1001, (1993). Thus, the assay for inhibition of NGF biological activity entail culturing NGF responsive cells with NGF plus an analyte, such as a candidate anti-NGF antibody and a candidate NGF antagonist. After an appropriate time the cell response will be assayed (cell differentiation, neurite outgrowth or cell survival).

The ability of a candidate NGF antagonist to block, reduce, inhibit, or neutralize a biological activity of NGF can also be carried out by monitoring the ability of the candidate agent to inhibit NGF mediated survival in the embryonic rat dorsal root ganglia survival bioassay as described in Hongo et al., Hybridoma 19:215-227 (2000).

### COMPOSITIONS FOR USE IN THE METHODS OF THE INVENTION

The compositions used in the methods of the invention comprise an effective amount of an NGF antagonist (such as an anti-NGF antibody). In one embodiment, the composition comprises one or more NGF antagonist. In another embodiment, the composition comprises one or more NGF antagonist selected from any one or more of the following: an antagonist (e.g., an antibody) that binds (physically interacts with) NGF, an antagonist that binds to an NGF receptor (such as TrkA receptor and/or p75 receptor), and an antagonist that reduces (impedes and/or blocks) downstream NGF receptor signaling. In other embodiments, an NGF antagonist inhibits (reduces) NGF synthesis, production or release. In some embodiments, the NGF antagonist is selected from one or more of the following: an anti-NGF antibody, an anti-sense molecule directed to an NGF (including an anti-sense molecule directed to a nucleic acid encoding NGF), an anti-sense molecule directed to a NGF receptor, an NGF inhibitory compound, an NGF structural analog, a dominant-negative mutation of a TrkA receptor that binds an NGF, a TrkA immunoadhesin, an anti-TrkA antibody, an anti-p75 antibody and a kinase inhibitor. In another embodiment, the NGF antagonist is an anti-NGF antibody. In other embodiments, the NGF antibody recognizes human NGF. In still other embodiments, the anti-NGF antibody is humanized (such as antibody E3 described herein). In still other embodiment, the NGF antibody comprises a constant region that does not trigger antibody-mediated lysis or ADCC.

In some embodiments, the composition comprises the humanized anti-NGF antibody E3 described herein. In other embodiments, the composition comprises an anti-NGF antibody comprising one or more CDR(s) of antibody E3 (such as one, two, three, four, five, or, in some embodiments, all six CDRs from E3).

It is understood that the compositions can comprise more than one NGF antagonist. For example, a composition can comprise more than one member of a class of NGF antagonist (e.g., a mixture of anti-NGF antibodies that recognize different epitopes of NGF), as well as members of different classes of NGF antagonists (e.g., an anti-NGF antibody and an NGF inhibitory compound). Other exemplary compositions comprise more than one anti-NGF antibodies that recognize the same epitope(s), different species of anti-NGF antibodies that bind to different epitopes of NGF, or different NGF inhibitory compounds.

The composition used in the present invention can further comprise pharmaceutically acceptable carriers, excipients, or stabilizers (Remington: The Science and practice of Pharmacy 20th Ed. (2000) Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The compositions described herein may contain additional compounds known to be useful for the treatment of cardiac arrhythmia, including but not limited to: beta-blockers, ACE inhibitors, aldosterone receptor blockers, amiodarone, potassium channel blockers such as d-sotalol and dofetilide, calcium channel blockers (e.g., nifidipine), and sodium channel blockers. Cardioprotective agents, antibiotics, antiviral agents, or thrombolytic agents (e.g., streptokinase, tissue plasminogen activator, or recombinant tissue plasminogen activator) may also be contained in the composition. Such molecules are suitably present in combination in amounts that are effective for the purpose intended. The NGF antagonist and compositions thereof can also be used in conjunction with other agents that serve to enhance and/or complement the effectiveness of the agents, including but not limited to: beta-blockers, ACE inhibitors, aldosterone receptor blockers, amiodarone, potassium channel blockers such as d-sotalol and dofetilide, calcium channel blockers (e.g., nifidipine), sodium channel blockers, cardioprotective agents, antibiotics, antiviral agents, or thrombolytic agents (e.g., streptokinase, tissue plasminogen activator, or recombinant tissue plasminogen activator).

Kits

The invention also provides kits for use in the instant methods. Kits of the invention include one or more containers comprising an NGF antagonist (such as an antibody described herein) and instructions for use in accordance with any of the methods described herein, such as methods of treating, preventing, ameliorating and/or reducing incidence of a NGF-associated cardiac arrhythmia; methods of preventing or reducing risk of death due to cardiac arrhythmia; methods of enhancing cardiac function in individuals in need thereof; methods of enhancing cardiac function and/or decreasing sympathetic innervation or sympathetic activity in an individual at risk of developing a cardiac arrhythmia; methods of preventing SCD associated with (and/or due to) cardiac arrhythmia; methods of delaying development and/or preventing death resulting from cardiac arrhythmia, including preventing SCD; and methods of reducing risk of development or progression of a cardiac arrhythmia in an individual at risk of developing cardiac arrhythmia The instructions may also comprise a description of selecting an individual suitable for treatment based on identifying whether that individual has an arrhythmia and/or is at risk of developing an arrhythmia In some embodiments, the instructions comprise description of administering an NGF antagonist to an individual at risk of developing an arrhythmia. In still other embodiments, the instructions comprise description of administering an NGF antagonist to improve cardiac function in an individual at risk of a symptom of an arrhythmia. In another embodiment, the instructions comprise description of administering an NGF antagonist to an individual at risk of SCD (such as an individual with MI or history of MI).

In some embodiments, the kit comprises an anti-NGF antibody. In other embodiments, the anti-NGF antibody is the antibody E3 as described herein. In other embodiments, the anti-NGF antibody comprises one or more CDR(s) of antibody E3 (such as one, two, three, four, five, or, in some embodiments, all six CDRs from E3).

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle).

The instructions relating to the use of an NGF antagonist generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, the kit comprises a container and a label or package insert(s) on or associated with the container. The container holds a composition which is effective for any of the methods described herein, such as treating an NGF-associated cardiac arrhythmia. At least one active agent in the composition is an NGF antagonist, such as an anti-NGF antibody. The container may further comprise a second pharmaceutically active agent. Kits may optionally provide additional components such as buffers and interpretive information.

Administration of an NGF Antagonist and Assessment of Treatment

The NGF antagonist can be administered to an individual via any suitable route. For example, the NGF antagonist can be administered orally, intravenously, subcutaneously, sublingually, intraarterially, intrasynovially, intravescicular (such as via the bladder), intramuscularly, intracardiacly, intrathoracicly, intraperitoneally, intraventricularly, sublingually, by inhalation, by suppository, and transdermally. It can be administered orally, for example, in the form of tablets, troches, capsules, elixirs, suspensions, syrups, wafers, lollypops, chewing gum or the like prepared by art recognized procedures. It should be apparent to a person skilled in the art that the examples described herein are not intended to be limiting but to be illustrative of the techniques available.

In one embodiment, an NGF antagonist is administered via site-specific or targeted local delivery techniques. Examples of site-specific or targeted local delivery techniques include local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211; U.S. Pat. No. 5,981,568.

In one embodiment, a catheter is placed into the coronary artery of an individual and an effective amount of an NGF antagonist is injected into the heart of the individual, for example, into a coronary artery. In some embodiments, the NGF antagonist is injected into an atria, ventricle, or the pericardium. The injection can be repeated as needed. Injection can also be by other routes, including, but not limited to, by catheter via arterial angiography, intracoronary injection, in a cardioplegic solution by the aortic route, and injection into the sympathetic trunk or ganglion.

In another embodiment, an NGF antagonist is administered by systemic infusion directly into the heart (such as into the myocardium, pericardium or a heart chamber (s)) via an osmotic pump.

Various formulations of NGF antagonists such as an anti-NGF antibody may be used for administration. In some embodiments, NGF antagonist(s) such as an anti-NGF antibody may be administered neat. In some embodiments, the composition comprises anti-NGF antibody and a pharmaceutically acceptable excipient, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Generally, these agents are formulated for administration by injection (e.g., intraperitoneally, intravenously, subcutaneously, intramuscularly, etc.). Accordingly, these agents are preferably combined with pharmaceutically acceptable vehicles such as saline, Ringer's solution, dextrose solution, and the like. The particular dosage regimen, i.e., dose, timing and repetition, will depend on the particular individual and that individual's medical history. Generally, a dose of at least about 3 μg/kg body weight, at least about 10 μg/kg body weight, at least about 30 μg/kg body weight, at least about 100 μg/kg body weight, at least about 250 μg/kg body weight, at least about 300 μg/kg body weight, at least about 750 μg/kg body weight, at least about 1 mg/kg body weight, at least about 3 mg/kg body weight, at least about 5 mg/kg body weight, at least about 10 mg/kg body weight, or at least about 30 mg/kg body weight, or more is administered.

An anti-NGF antibody can be administered by any means known in the art, including injection (e.g., intraperitoneally, intravenously, subcutaneously, intramuscularly, etc.) including injection directly into the coronary artery. An anti-NGF antibody can also be administered via inhalation, as described herein. Generally, for administration of anti-NGF antibody, an initial candidate dosage can be about 2 mg/kg. For the purpose of the present invention, a typical daily dosage might range from about any of 3 μg/kg to 30 μg/kg to 1 mg/kg to 30 mg/kg to 100 mg/kg or more, depending on the factors mentioned above. For repeated administrations over several days or longer, depending on the condition, the treatment is sustained until a desired suppression of disease symptoms occurs or until sufficient therapeutic levels are achieved to reduce the risk of arrhythmia. An exemplary dosing regimen comprises administering an initial dose of about 2 mg/kg, followed by a weekly maintenance dose of about 1 mg/kg of the anti-NGF antibody, or followed by a maintenance dose of about 1 mg/kg every other week. However, other dosage regimens may be useful, depending on the pattern of pharmacokinetic decay that the practitioner wishes to achieve. The progress of this therapy is easily monitored by conventional techniques and assays.

In general, when it is not an antibody, an NGF antagonist according to the invention may be administered at the rate of 0.1 to 300 mg/kg of the weight of the patient divided into one to three doses, or as disclosed herein. For an adult patient of normal weight, doses ranging from about 0.3 to 5.00 mg/kg (or more) may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present invention, the appropriate dosage of an NGF antagonist will depend on the NGF antagonist(s) (or compositions thereof) employed, the type of cardiac arrhythmia to be treated, the severity and course of the disease, whether the agent is administered for preventive or therapeutic purposes, previous therapy, the patient's clinical history and response to the agent, and the discretion of the attending physician. Typically the clinician will administer an NGF antagonist, such as an anti-NGF antibody, until a dosage (and/or serum plasma level) is reached that achieves the desired result. The frequency of dosing can change over time.

Empirical considerations, such as the half-life, generally will contribute to the determination of the dosage. Antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of one or more symptoms associated with cardiac arrhythmia described herein. Alternatively, sustained continuous release formulations of anti-NGF antibodies may be appropriate. Various formulations and devices for achieving sustained release are known in the art.

In one embodiment, dosages for an NGF antagonists may be determined empirically in individuals who have been given one or more administration(s) of an anti-NGF antagonist as described herein. Individuals are given incremental dosages of an agent which inhibits NGF, e.g., anti-NGF antibody. To assess efficacy of an NGF antagonist, an indicator of cardiac arrhythmia can be followed.

Administration of an NGF antagonist in accordance with the method in the present invention can be continuous or intermittent, depending, for example, upon the recipient's physiological condition, whether the purpose of the administration is therapeutic or prophylactic, and other factors known to skilled practitioners. The administration of an NGF antagonist may be essentially continuous over a preselected period of time or may be in a series of spaced dose, e.g., either before, during, or after developing symptoms of cardiac arrhythmia; before and during; before and after; during and after; or before, during, and after developing symptoms of arrhythmia. The administration of an NGF antagonist may also occur during and/or after a predisposing event, such as MI. Administration of an NGF antagonist can be chronic or acute.

Other formulations include suitable delivery forms known in the art including, but not limited to, carriers such as liposome. See, for example, Mahato et al. (1997) Pharm. Res. 14:853-859. Liposomal preparations include, but are not limited to, cytofectins, multilamellar vesicles and unilamellar vesicles.

In some embodiments, more than one NGF antagonist, such as an antibody, may be present. The antagonists can comprise more than one member of a class of NGF antagonist (e.g., a mixture of anti-NGF antibodies that recognize different epitopes of NGF), as well as members of different classes of NGF antagonists (e.g., an anti-NGF antibody and an NGF inhibitory compound). Other exemplary compositions comprise more than one anti-NGF antibodies that recognize the same epitope(s), different species of anti-NGF antibodies that bind to different epitopes of NGF, or different NGF inhibitory compounds. At least one, at least two, at least three, at least four, at least five different NGF antagonists can be present. Generally, those NGF antagonists have complementary activities that do not adversely affect each other.

An NGF antagonist can also be used in conjunction with other agents that serve to enhance and/or complement the effectiveness of the NGF antagonist. Examples of such agents include, but are not limited to, beta-blockers, ACE inhibitors, aldosterone receptor blockers, amiodarone, potassium channel blockers such as d-sotalol and dofetilide, calcium channel blockers (e.g., nifidipine), sodium channel blockers, cardioprotective agents, antibiotics, antiviral agents, or thrombolytic agents (e.g., streptokinase, tissue plasminogen activator, or recombinant tissue plasminogen activator)

Therapeutic formulations of an NGF antagonist (such as an antibody) used in accordance with the present invention are prepared for storage by mixing an NGF antagonist (such as an antibody) having the desired degree of purity with optional pharmaceutically acceptable carriers, excipients or stabilizers (Remington, The Science and Practice of Pharmacy 20th Ed. Mack Publishing (2000)). In some embodiments involving anti-NGF antibodies, pharmaceutically acceptable carriers, excipients or stabilizers are in the form of lyophilized formulations, or aqueous solutions or suspensions. Acceptable carriers, excipients, or stabilizers are nontoxic to recipients at the dosages and concentrations employed, and may comprise buffers such as phosphate, citrate, and other organic acids; salts, such as sodium chloride, antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosacchandes, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the NGF antagonist (such as an antibody) are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-Lglutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3hydroxybutyric acid.

The formulations to be used for ill vivo administration must be sterile. This is readily accomplished by, for example, filtration through sterile filtration membranes. Therapeutic anti-NGF antibody compositions are generally placed into a container having a sterile access port, for example, an intravenous solution bag or vial having a stopper pierceable by a hypodermic injection needle.

The NGF antagonist, such as an anti-NGF antibody, is administered to a individual in accord with known methods, such as intravenous administration, e.g., as a bolus or by continuous infusion over a period of time, by intramuscular, intraperitoneal, subcutaneous, inhalation, oral, or topical routes. Commercially available nebulizers for liquid formulations, including jet nebulizers and ultrasonic nebulizers are useful for administration via inhalation. Liquid formulations can be directly nebulized and lyophilized powder can be nebulized after reconstitution. Alternatively, an NGF antagonist, e.g., anti-NGF antibody, can be aerosolized using a fluorocarbon formulation and a metered dose inhaler, or inhaled as a lyophilized and milled powder.

Treatment efficacy can be assessed by several methods. Fogel et al., Crit Care Med., 28 (10 Suppl.):165-169 (2000). For example, serial electrophysiologic-pharmacologic testing can be used. In the testing, individuals are typically studied in the baseline state to determine their inducibility of ventricular tachycardia. After adequate administration of an NGF antagonist, repeat EP testing can then be performed to assess the suppression of inducible ventricular tachycardia. The drug efficacy can also be judged by quantitative suppression of spontaneous arrhythmia, as proposed by the Lown group. Graboys T B, et al., Am. J. Cardiol., 50:437443 (1982). Assessment may also be made by monitoring clinical signs such as heart rate variability (using, for example a Holter monitor), ECG signals, left ventricular ejection fractions (LVEF), electrophysiological responses, or molecular changes.

The following Examples are provided to illustrate but not limit the invention.

### EXAMPLE

### Example 1

### Effect of an NGF Antagonist on Dogs with Induced MI

Fifteen adult canines are chosen, nine for the experiment and six for the control. In each adult canine, an MI and AV block are created using methods disclosed in PCT Publication No. WO 01/62334. Specifically, an MI is created by ligating the left anterior descending coronary artery just below the first diagonal branch. An AV block is created by radiofrequency catheter ablation.

An implantable cardiovascular-defibrillator (ICD, Guidant model 1762 or 1810) is implanted within the subject, and appropriate leads are connected to the heart of the subject. The ICD is programmed to the monitor-only mode with a back-up pacing rate of 40 bpm. During follow up, the ICD declares VT episodes once the ventricular rate exceeds 100 bpm for 8 to 10 beats.

Immediately after the MI, an NGF antagonist (such as an anti-NGF antibody or a canine TrkA immunoadhesin) is administered via IV injection. If anti-NGF antibodies are used, the dosage is approximately 1 0 mg anti-NGF antibody per kg body weight. Saline solution is administered in the same fashion to the six control dogs. The dogs are allowed to recover for up to 3 months and are closely monitored to document VT episodes, VF episodes, and SCD.

Immunohistochemical studies are done to document the reduction of sympathetic hyperinnervation in the ventricular myocardium. Left ventricular tissues from the edge of the posterior papillary muscle, the anterior papillary muscle, and the interventricular septum of the middle sections are used for immunocytochemical studies. The tissues in the AV nodal region are also sectioned for immunohistochemical studies. The nerve markers tyrosine hydroxylase (TH), synaptophysin (SYN), and growth-associated protein 43 (GAP43) are stained and the density of innervation assessed by standard serological methods.

Student's t tests are used to compare the means between the two groups. To quantify the periodic structure of the frequency of occurrence of VT, single and double harmonic regression models are fitted to the data. The null hypothesis is rejected at a value of p<0.05.

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention.

We claim:

1. A method of treating a cardiac arrhythmia in an individual comprising administering an effective amount of an NGF antagonist.

2. The method of claim 1, wherein said cardiac arrhythmia comprises sustained ventricular tachycardia.

3. The method of claim 1, wherein said cardiac arrhythmia comprises non-sustained ventricular tachycardia.

4. The method of claim 1, wherein said cardiac arrhythmia comprises ventricular fibrillation.

5. The method of claim 1, wherein said cardiac arrhythmia is associated with sudden cardiac death.

6. The method of claim 1, wherein the NGF antagonist is an anti-NGF antibody.

7. The method of claim 6, wherein the anti-NGF antibody binds to human NGF.

8. The method of claim 6, wherein the binding affinity of the anti-NGF antibody is about 0.1 nM or less than about 0.1 nM.
